(19) United States
(12) Patent Application Publication
(10) Pub. No.: US 2019/0080442 A1
(43) Pub. Date: Mar. 14, 2019

(54) SYSTEM AND METHOD FOR IMAGE GUIDED TRACKING TO ENHANCE RADIATION THERAPY

(71) Applicants: The Charles Stark Draper Laboratory, Inc., Cambridge, MA (US); Brigham and Women's Hospital, Inc., Boston, MA (US)

(72) Inventors: Andrew A. Berlin, Lexington, MA (US); Christopher L. Williams, PhD, Cambridge, MA (US); Ross Berbeco, PhD, Cambridge, MA (US)

(21) Appl. No.: 15/912,205

(22) Filed: Mar. 5, 2018

Related U.S. Application Data
(60) Provisional application No. 62/467,225, filed on Mar. 5, 2017.

(51) Int. Cl. G06T 5/50; A61N 5/10; G06T 7/254

(52) U.S. Cl. CPC G06T 5/50; A61N 5/1067; G06T 7/254; G06T 2207/10028; G06T 2207/10081; G06T 2207/20208; G06T 2207/20224; G06T 2207/30101; G06T 2207/20221

(57) ABSTRACT

This invention provides a system and method that allows the utilization of computer vision system techniques and processes, such as multi-layer separation and contrast mapping, to enhance the detectability of an imaged tumor, opening the door to real-time tumor tracking and/or modulation of a treatment radiation beam so as to maximize the radiation dosage applied to the tumor itself while minimizing the dosage received by surrounding tissues. The techniques and processes also permit more accurate assessment of the level of radiation dosage delivered to the tumor. An image processor receives the image data from the detector as a plurality of image frames, and performs contrast stretching on the image frames to resolve features. A motion analysis module compares static and dynamic features in the contrast-stretched image frames to derive layers of features. The image frames are output as enhanced image frames. The output can be used to guide the beam.

### RELATED APPLICATION

This application claims the benefit of co-pending U.S. Provisional Application Ser. No. 62/467,225, entitled SYSTEM AND METHOD FOR IMAGE GUIDED TRACKING TO ENHANCE RADIATION THERAPY, filed Mar. 5, 2017, the teachings of which are expressly incorporated herein by reference.

### FIELD OF THE INVENTION

This invention relates to medical imaging for use in therapeutic treatments and more particularly to guidance of radiation therapy systems via medical imaging for use in treatment of tumorous tissue.

### BACKGROUND OF THE INVENTION

Radiation therapy is a desirable, non-surgical technique for treating certain forms of cancerous growths and/or tumors. The radiation source—for example an x-ray or proton beam is focused upon the region of the tissue that contains the tumor. Typically, medical imaging, in the form of (e.g.) an MRI, CT scan and/or PET scan localizes the region containing the tumor in advance of the therapy procedure. In this manner, the beam can be more accurately concentrated on the diseased tissue.

In the case of an x-ray photon beam, the patient is placed between the X-ray source and a flat panel detector that allows an image of the tissue to be acquired during irradiation. This assists in guiding the beam to some extent.

By way of background, x-ray based radiation therapy is delivered by accelerating high-energy electrons and generating bremsstrahlung x-rays through directing the beam at a high atomic number target. These photons are collimated, and directed at the target. Proton beam therapy employs a cyclotron or synchrotron to energize protons. Protons are extracted from the cyclotron or synchrotron and directed with magnetic fields to the tumor. The depth to which the protons in the beam penetrate the body/tissue is related to their relative energy which can be accurately controlled to match the position of the tumor. Protons deliver the majority of their energy at a very narrow area within the body. This unique dose delivery property of protons is known as the Bragg Peak. The Bragg Peak area can be manipulated to provide the desired radiation dose to the tumor itself without any exit dose beyond the tumor. Advantageously the radiation dose to the uninvolved normal tissues surrounding the tumor is reduced by use of a proton beam in treatment. The shaping of the proton beam can be controlled by magnetically scanning across the tumor volume and by adjusting a multi-leaf collimator.

When treating ailments such as lung cancer using radiation therapy, using (e.g.) an x-ray photon or a proton beam, it is desirable to localize the radiation delivery to the tumor location, so as to minimize the destruction of surrounding tissue. However, medical imaging technologies (i.e. x-ray imaging and/or proton beam) have disadvantages in that energy from objects of varying density within the patient's body such as ribs partially, but not completely, obscure objects of interest such as tumors. This renders the resulting image unclear and increases the difficulty for the user in guiding the beam within the patient. In addition, patients and their internal tissues tend to move during treatment, due to a variety of factors such a respiration, blood flow, muscle contractions, etc. These movements render it difficult to maintain a therapeutic radiation beam on target at all times, even if the beam is initially precisely aligned with the treatment field.

One approach to addressing the above disadvantages is to subtract out the ribs by acquiring two (or more) pictures utilizing at least two different energy levels of radiation, one strong enough to penetrate the ribs, and one less strong. The two images are subtracted to identify the features that lie beyond the ribs. See, Markerless Motion Tracking of Lung Tumors Using Dual-Energy Fluoroscopy, by Rakesh Patel, Joshua Panfil, Maria Campana, Alec M. Block, Matthew M. Harkenrider, Murat Surucu, and John C. Roeske, Medical Physics 42, 254 (2015); doi: 10.1118/1.4903892. However, this approach requires the continuous use of a dual energy radiation system (unavailable in most facilities) to achieve any sort of real-time (or near-real-time) imaging. Additionally, patient motion between the high energy and low energy beam exposure (understanding that current dual energy systems typically require significant time to change energy levels) will introduce undesirable artifacts to the subtracted image result.

Other approaches have been attempted for compensating for tumor movement during therapy. These various approaches are summarized in Tumour Movement in Proton Therapy: Solutions and Remaining Questions: A Review, by Dirk DeRuysscher et al., Cancers (2015), 7, 1143-1153; doi: 10.3390/cancers7030829. For the most part these solutions involve calculating an increased volume of tissue to irradiate to ensure that even in the presence of tumor motion, the tumor receives a sufficient dose of radiation. This can lead to undesirable outcomes, where the goal is to limit the degree of tissue exposure and concentrate exposure in the diseased (tumorous) tissue region.

Yet another approach is to gate the radiation beam to the respiratory cycle. Typically to the mid-point of the respiratory cycle. This can be accomplished by measuring the rate of breathing using a spirometer, tracking of the surface of the skin, or via tension belts that register breathing motion. Unfortunately patient's breathing cycles do not repeat in a very well-controlled manner over the duration of a radiation treatment, so these techniques, while helpful, are not sufficient to solve the problem of maintaining the beam on a desired position within the treatment field.

Thus, all of the above approaches have disadvantages in terms of equipment, time, and accuracy.

### SUMMARY OF THE INVENTION

This invention overcomes disadvantages of the prior art by providing a system and method that allows the utilization of computer vision system techniques and processes, such as multi-layer separation and contrast mapping, to enhance the detectability of an imaged tumor, opening the door to real-time tumor tracking and/or modulation of a treatment radiation beam so as to maximize the radiation dosage applied to the tumor itself while minimizing the dosage received by surrounding tissues. The illustrative techniques and processes also permit more accurate assessment of the level of radiation dosage delivered to the tumor.

In an illustrative embodiment, a system and method applies a radiation beam for therapy of an internal region of a body of the patient that passes through the region and is received by a detector to generate images thereof. The system and method includes an image processor that receives the image data from the detector as a plurality of image frames, and that performs layer separation within the plurality of image frames. A motion analysis module compares static and dynamic features in the image frames to derive features in the separated layers. The image frames are based on the layers of features being provided to an output as enhanced image frames. A feature detection module applies vision system tools to the features in the enhanced image frames to identify information contained therein. Illustratively, the information is used to track motion of the features versus time. The tracked motion information is provided to a beam positioner that changes a position or orientation of the radiation beam based on a degree and direction of tracked motion, and/or it is provided to an actuation system that moves or restrains the patient to maintain the radiation beam at a desired position in the region. The radiation beam can be comprised of, at least one of, x-rays, gamma rays, a proton beam, a stereotactic body radiation therapy (SBRT) source, a three-dimensional conformal radiation therapy (3D-CRT) source, an intensity-modulated radiation therapy (IMRT) source, and a radiosurgery source. Illustratively, an analysis module compares anatomical features from scans obtained at a time remote from the use of the radiation beam to features output in the enhanced image frames. The scans can provide CT-based, MRI-based, PET-based, or other medical imagery-based, pre-treatment images and the analysis module can be arranged to generate fused images comprising the pre-treatment images and the enhanced image frames. Also, the fused images can include at least one of information and depth relative to the features. Illustratively, the radiation beam can be arranged on a continuously rotating structure that encircles the patient to emit the beam around a 360-degree perimeter thereof. A display processor can be arranged to display to a user a display model that is derived from the enhanced images, including information useful in diagnosing the imaged region or administering treatment to the imaged region. The information can define at least one of (a) shading and (b) color-coding of areas of the display model to characterize a degree of exposure to the radiation beam over time, and/or can be defined as at least one of (a) a graph, (b) a histogram, and (c) a plot that characterized exposure to the radiation beam versus time across the region. In embodiments, the display processor can be arranged to perform contrast stretching on the plurality of image frames to assist in visually resolving image features therein. The radiation beam, which can be arranged to rotate about the patient, can also include a tracking process that accounts for motion of the beam with respect to the region in generating the enhanced image frames. In embodiments, a fusion module integrates pre-treatment information with the enhanced images to assist in defining a subject of interest in the region relative to other information therein. More particularly, the subject of interest can be a tumor, and the pre-treatment information identifies a layer of the enhanced images containing the tumor. In embodiments, a pattern-matching process operates on the pre-treatment information, in the form of pre-treatment images, and the enhanced images, based upon matching, for at least one region of the pre-treatment images and the enhanced images, at least of (a) estimated volume, (b) shape, (c) texture, (d) intensity histogram, (e) edges, (f) velocity, and (g) projected area. Illustratively, the motion analysis module can be arranged to identify and manipulate instances of occlusion or saturation in the plurality of image frames. Embodiments of the system and method can include an image compositing process that is arranged to fill in items of the enhanced images that are missing based upon the occlusion or saturation. The system and method can also include an image processor, which selectively applies at least one of amplification and tone-correction to the enhanced images and/or an intensity adjustment process that compensates for intensity reduction in a subject image frame of the plurality of image frames based upon loss of signal energy due to items present in image frames overlying or underlying the subject image frame.

In an illustrative embodiment, a method for treating and imaging a tissue region in a patient, using a radiation treatment beam that passes through the region and is received by a detector, is provided. The method includes the steps of receiving the image data from the detector as a plurality of image frames, analyzing static and dynamic features in the image frames to derive layer-separated images of the features, and outputting the image frames based on the layers of the features as enhanced image frames. Illustratively, the method can include performing contrast stretching on at least one of the layer-separated images to resolve features therein.

### BRIEF DESCRIPTION OF THE DRAWINGS

The invention description below refers to the accompanying drawings, of which:

FIG. 1 is a diagram of an exemplary radiation treatment arrangement including an exemplary proton beam and imaging array and associated controller and processor, according to an illustrative embodiment;

FIG. 1A is a flow diagram of an exemplary procedure for generating a high-dynamic range (HDR) image through segmentation and enhancement of raw, acquired image frames for use with the system and method herein,

FIG. 1B is a flow diagram of a light/pixel data segmentation module for use with the exemplary procedure of FIG. 1A;

FIG. 2 is a block diagram showing the operational steps of the overall process in for enhancing images, detecting features and controlling a radiation (e.g. x-ray proton) therapy beam according to an illustrative embodiment;

FIG. 3 is a display of an image of an exemplary proton beam image provided by the arrangement of FIG. 1, prior to image processing performed herein;

FIG. 4 is display showing selective contrast mapping according to an illustrative embodiment applied to the exemplary image of FIG. 3;

FIG. 5 is a display showing a fraction of the image of FIG. 4 whose intensity is free of variation, i.e. the minimum intensity present in each element of the proton beam image stream;

FIG. 6 is a display showing a comparison of images based upon the image of FIG. 4, wherein the fraction of the x-ray beam image whose intensity is free of variation, in which the left image is shown after undergoing local contrast enhancement, and the right image is the fraction of the proton beam image whose intensity is free of variation, after undergoing constraint-based subtraction to adjust contrast, in which a constraint was manually applied, indicating that the darker material that has a different texture is to be ignored;

FIG. 7 is a display, based upon the exemplary image of FIG. 3 of a typical frame of the portion of the x-ray beam image whose intensity is free of significant variation, i.e. the temporally dynamic portion of the image, whereby blood vessels and other dynamic features within the imaged tissue become apparent to a viewer;

FIG. 8 is a display of a comparison of exemplary left and right images based upon the image of FIG. 3, in which the left image is the full frame that served as the starting point for motion-based processing, having undergone only basic contrast stretching, and thereby provides a reference, and the right image is a frame from a blended video that amplifies the dynamic portion but also includes (e.g.) 30% of the static portion, for reference;

FIG. 9 is a block diagram of a process for modelling image data for use in monitoring and adjusting treatment using a treatment beam in both a manually and automatically operated treatment beam application procedure; and

FIG. 10 is a diagram of a series of image frames of an imaged patient region, and a resulting, displayed, color-coded model based on exposure time as a result of patient movement (breathing, voluntary and involuntary motion, etc.) according to an embodiment.

### DETAILED DESCRIPTION

I. System Overview

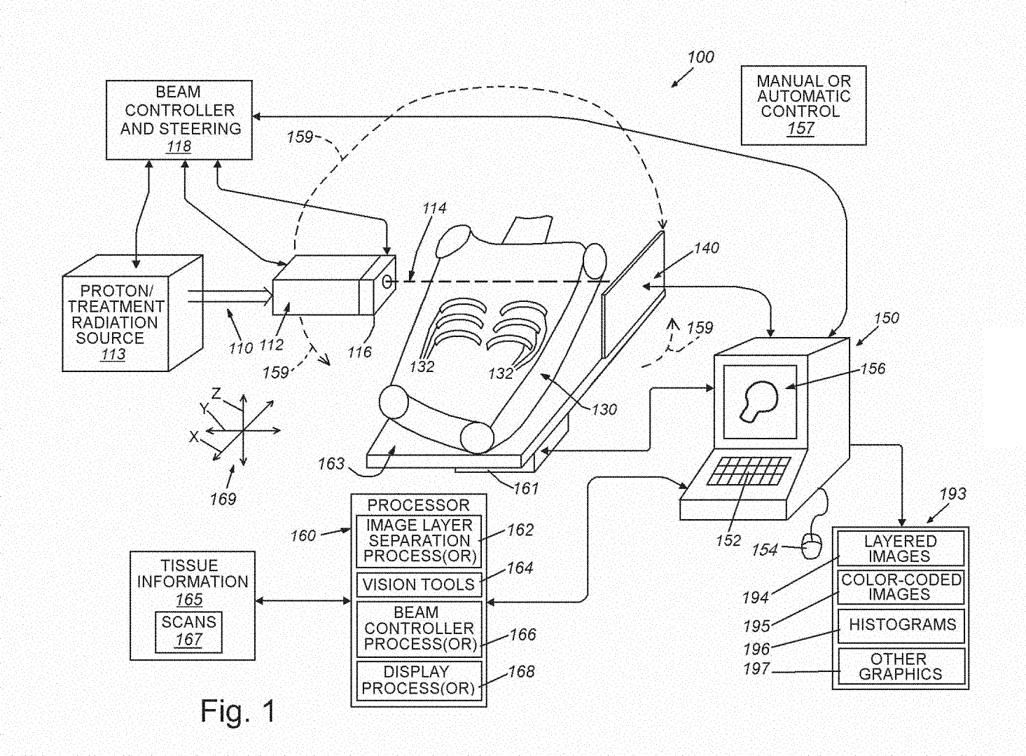

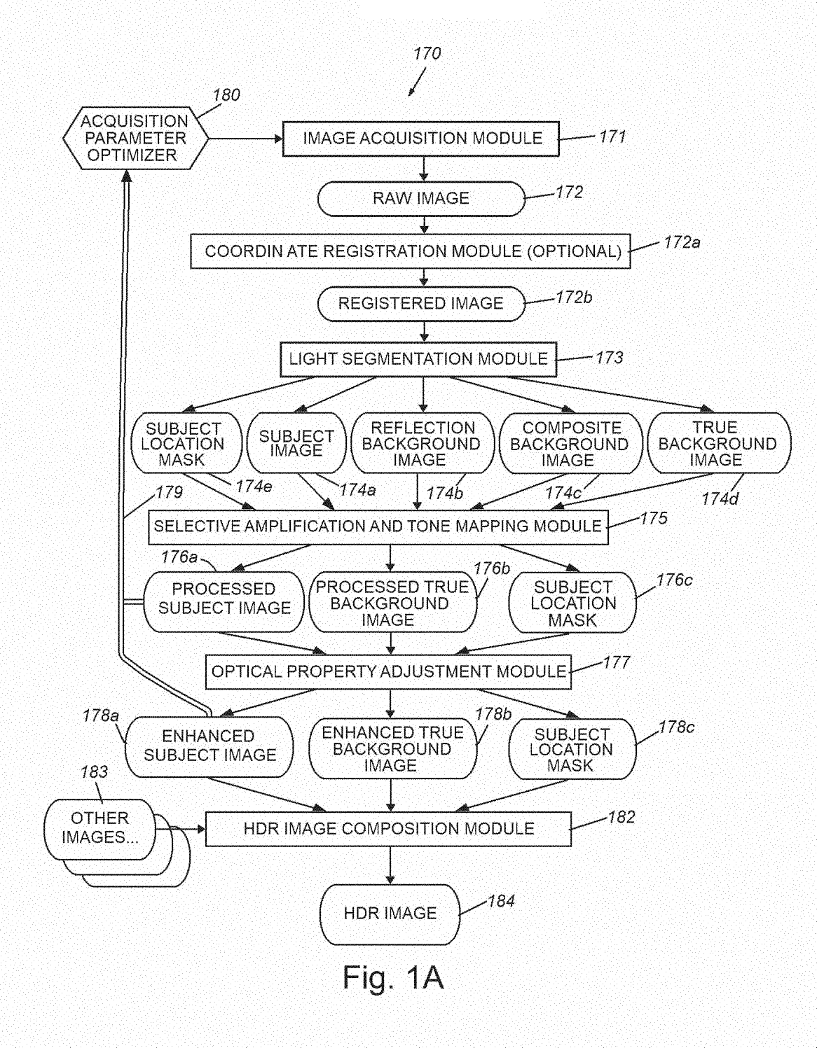

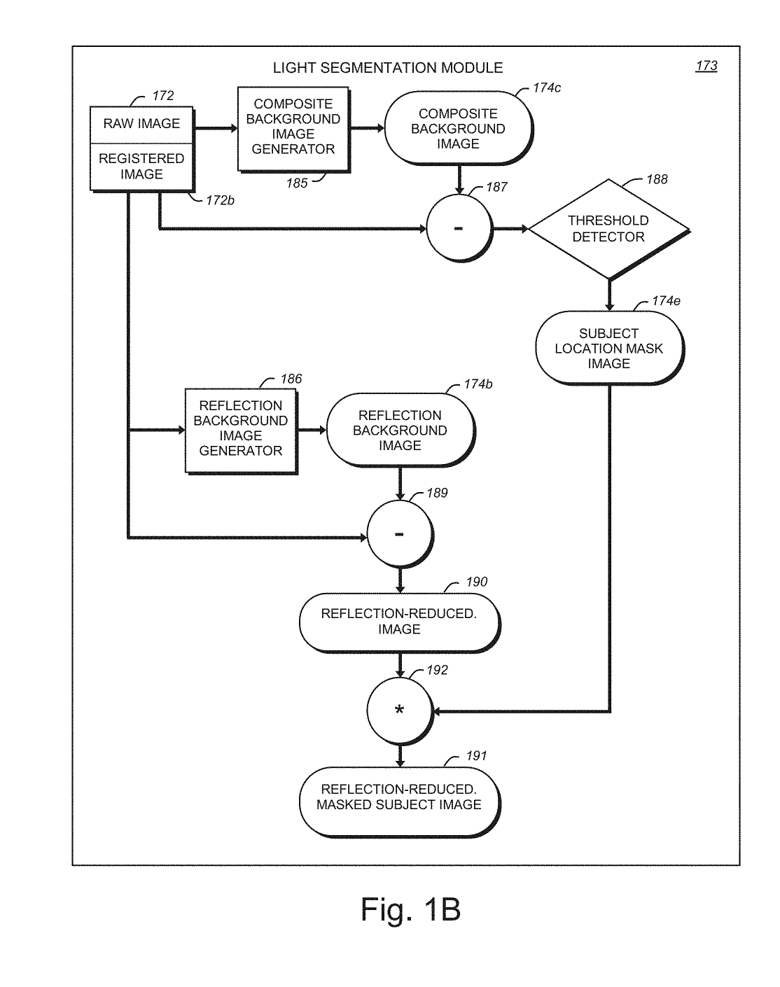

FIG. 1 shows a generalized overview of an exemplary arrangement 100 that includes a proton beam radiation therapy system 110 according to an embodiment. The radiation beam generating beam device 112 is shown schematically. In practice it includes a source 113 of therapeutic radiation (either an electron linear accelerator and target for x-ray photon-based therapy or a large proton-generating cyclotron or synchrotron assembly for proton-based therapy). The proton beam (dashed line 114) is aimed or formed into a desired shape and/or focus and aimed by a shaping/positioning assembly 116 (also termed an “aperture”). This can consist of a mask that mimics the shape of the treatment area or an array of controllable magnets. The system 110 is controlled by a controller 118 that can be conventional in design and capable of interfacing with an external control computer (described below). As shown, the controller generally operates the proton source, the device 112 and the shaping/positioning assembly 116.

The beam 114 is shown traversing an exemplary torso 130 of a patient requiring radiation therapy (for example, to treat a cancerous tumor in the lung, where it encounters ribs 132 and other potential, higher-density obstructions that can be problematic). The beam exits the torso and is received by a flat panel imager (FPI) 140 that is adapted to transmit received image data (e.g. in the form of grayscale intensity pixels) to a computing device 150. By way of non-limiting example, the computing device 150 can be implemented as a customized data processing device or as a general purpose computing device, such as a desktop PC, server, laptop, tablet, smartphone and/or networked “cloud” computing arrangement. The computing device 150 includes appropriate network and device interfaces (e.g. USB, Ethernet, WiFi, Bluetooth®, etc.) to support data acquisition from external devices, such as the FPI 140 the proton beam controller 118. These network and data interfaces also support data transmission/receipt to/from external networks (e.g. the Internet) and devices as appropriate to transmit and receive data to remote locations, users and devices. The computing device 150 can include various user interface (UI) and/or graphical user interface (GUI) components, such as a keyboard 152, mouse 154 and/or display/touchscreen 156 that can be implemented in a manner clear to those of skill. The display 156 can provide images of tissue via the FPI as described below.

The computing device 150 includes a processor 160 according to embodiments herein, which can be implemented in hardware, software, or a combination thereof. The processor 160 receives data from the FPI 140 and the beam controller 118 and provides data thereto. In an embodiment, the processor 160 is organized into various processes or functional modules that can be organized in a variety of ways. By way of non-limiting example, the processor 160 includes an image layer separation process 162 that allows features in the FPI-generated image to be resolved according to the system and method herein. The processor 160 can also includes various machine/computer vision tools and processes 164, such as edge detectors, blob analyzers, calipers, and the like for extracting and analyzing features in the image provided by the FPI to the processor 160. Additionally, the processor 160 can include a beam control module/process(or) 166 that interacts with the shaping/positioning assembly 116 via the controller 118 to selective activate/deactivate and direct the beam 114 based upon the image data processed and extracted by the modules 162 and 164.

A display process(or)/module 168 processes and generates image information related to imaged features for display on the depicted display 156, or another display, and/or to be stored for later display on a storage device. The display process(or) 168 also allows for merging and/or fusion of images as described below, as well as other functions related to presentation and arrangement of image date for display to the user (e.g. color coding of features, layers, etc.).

The processor 160 also interfaces with a local or remote store 165 of data/information related to the tissue being treated and imaged. This can include stored scans/images 167 of the treatment field (e.g. tumor) that have been obtained in previous sessions with the patient—for example, using CT, PET and MM scans, as well as x-ray and proton-beam imaging. This image data can assist the user and processor in localizing the treatment based on prior knowledge of the geometry and features present in the treatment field.

In general, reference can be made, by way of useful background information, to J. Rottmann, P. Keall and R. Berbeco, Real-time soft tissue motion estimation for lung tumors during radiotherapy delivery, Med Phys. 2013 September; 40(9): 091713; Published online 2013 August 26. doi: 10.1118/1.4818655. This publication discuss the problem of patient motion during radiotherapy treatment and techniques for estimating such motion. The novel solutions provided herein address such problems and enhance the effectiveness of radiotherapy.

II. Image Layer Separation

To perform the functions of the systems and methods herein, the embodiments make use of image layer separation techniques. These techniques allow an acquired image to be presented as a so-called “high dynamic range” (HDR) image that contains information useful in separating various feature sets for analysis, which would otherwise be obscured or occluded. A variety of techniques can be employed to generate a separated-layer image and/or HDR image. A basic technique can employ tone-mapping, including techniques such as white-balancing, brightness enhancement, contrast enhancement, color-correction, intensity-level shifting, etc., which are well-known to those of skill. In order to address certain issues that can reduce the effectiveness of a tone-mapping approach, such as reflectivity and glare in the image, tone-mapping can be applied preferentially to light from the subject relative to light from other sources (described below).

Another approach to generating an HDR image is to apply HDR photography techniques, which typically combine multiple different exposures of a scene. These techniques have proven to be helpful in providing added tonal detail in low-lit areas (where a longer-exposure image would typically be used), while simultaneously permitting detail to be shown without saturation in brightly-lit areas (where a shorter-exposure time image would typically be used). Combining portions of multiple images enables the HDR approach to achieve greater detail in a single image than would otherwise be possible, and can permit combinations of features that would otherwise not be visible in any single image to become visible in the composite image.

Other techniques for generating layer separated and/or HDR images of varying qualities are described in the literature and should be clear to those of skill.

An approach to generating an HDR image, by way of non-limiting example, is effective to image objects through other translucent objects such as tinted windows, selectively amplifying the fraction of the light at each pixel that was due to the subject of the photograph (e.g. a person sitting in a car behind tinted or glare-filled windows) without (free of) amplifying light at each pixel associated with optical reflections off of the tinted windows or light associated with the background. It uses the rate of change associated with motion of the person sitting in the car, which differs from the rate of change associated with the reflected objects or background, as a queue to separate out the various sources of light. See, by way of exemplary background information, published U.S. Patent Application No. 2017/0352131 A1, entitled SPATIO-TEMPORAL DIFFERENTIAL SYNTHESIS OF DETAIL IMAGES FOR HIGH DYNAMIC RANGE IMAGING, published Dec. 7, 2017. Medical imaging technologies, such as those derived from non-visible and/or high-energy radiation (e.g. x-ray and proton-beam) imaging, can exhibit a similar effect, in which energy from overlying objects such as ribs partially, but not completely, obscures objects/features of interest, such as tumors. Underlying objects can also provide a signal that is partially obscured by the feature of interest, such as a tumor. The resulting images are thereby a combination of the features of interest and other features that are undesired and confuse the overall view of the treatment field.

In an embodiment, the method for amplifying a subject sitting in a car, partially obscured by reflected light from the sky and by tonal shifts introduced by window tinting, can be applied to optimize an image of a tumor partially obscured by a rib. In this embodiment, the coordinate system for the reflection removal computation is chosen such that the rib remains stationary as the patient breathes. This can be achieved, for example, by applying an alignment algorithm to the sequence of image frames prior to further processing of the frame. With the rib stationary in this aligned coordinate system, it blocks a portion of the radiation beam, creating a light (or other differing contrast) region on the resulting acquired image, corresponding to the radioopacity of the rib, analogous to the way that reflected light from the sky bouncing off of an automobile window adds light to an image that gets mixed with light from the subject. Additionally, the rib reduces the amount of illumination of the tumor itself, thereby reducing the relative intensity of the tumor, analogous to the way that tinting on a window reduces the amount of light illuminating a subject sitting in an automobile. So the algorithm developed for the automobile application, described below, may be directly applied to imaging of signal arising from a tumor mixed with signal arising from a rib, with the overlying rib acting as the reflective/tinted window and the tumor acting as the subject that moves around relative to the rib as the patient breathes.

Since the rib is composed primarily of solid tissue (non-deformable), even as the patient breathes, its radioopacity signature (provided that rib remains within the field of view) remains relatively constant. Thus, in an embodiment the initial alignment of the frames to create a coordinate system in which the rib is stationary may be achieved by a template matching approach that applies rigid deformation in a manner that maximizes the correlation of the image from frame to frame. In a more computationally complex embodiment, the coordinate system can be locally adjusted via deformable registration mechanisms to account for any out-of-plane motion of the rib, effectively maximizing correlation on a region-by-region or hierarchical basis, in addition to or instead of seeking to align on the overall images.

With reference particularly to FIG. 1A, a block diagram of an exemplary procedure 170 for generating an HDR image is shown in further detail. This procedure 170 receives an image from an acquisition module 171 (which can be any 2D or 3D imaging device in this embodiment). The raw 2D or 3D image 172 is forwarded to a light segmentation module 173 that separates the light associated with the subject from the light associated with the “true background”, which is the portion of the light signal that gets occluded by the subject, and the “reflective background”, which is the portion of the light signal that becomes mixed with the subject's light signal. As such, the module 173 generates a subject image 174a, reflection background image 174b, composite background image 174c, true background image 174d, and associated subject location mask—which locates and delineates the subject in the scene 174e.

As described above, the raw image 172 is optionally processed by a coordinate registration module 172a, which is arranged to map the coordinate system employed by the process so as to keep the primary object to be subtracted, such as a rib, stationary across multiple image frames. In this manner, the rib remains still (analogously to the above-described, exemplary car windows), and the tumor is allowed to move between image frames. The resulting registered image 172b is transmitted to the light-segmentation module 173 in this exemplary (optional) embodiment. The mapping of a registered image 172b, via an associated registration module 173, may be omitted if the operative light segmentation strategy, differs from the illustrative pixel based averaging described herein. For example, blocks 172a and 172b can be omitted where the light segmentation strategy entails measurement of velocity at each point.

With reference also to FIG. 1B, the operation of the exemplary light segmentation module 173 is shown in further detail. Based on the input raw image 172 or registered image 172b (also termed “raw or registered image” (or image data)), the composite background image 174c is modeled using a composite background image generator 185. The composite background includes light from both the reflection background and light from the true background. The module 173 also generates the reflection background image 174b by operating a reflection background image generator 186 on the raw or registered image (which can also be termed the “current” image frame) 172 or 172b. The module 173 identifies the location of the subject in the image to produce the mask image 174e. This is accomplished by subtracting (operator 187) the composite background image 174c from the raw or registered image 172 or 172b. A threshold detector 188 is also employed against the subtracted image to produce the final mask, which can define a binary image with the subject appearing as one binary value (1/0) and the background appearing as the other binary value (0/1). The reflection background image 174b is also subtracted (operator 189) from the raw or registered image 172 or 172b to generate a reflection-reduced image 190. A reflection-reduced, masked subject image 191 is then generated by isolating the pixels/regions of the current frame that contain the subject using the mask 174e (via operator 192) and subtracting values of the reflection reduced background image 190 from the isolated pixels/regions. Note that the module 173 can refrain from generating the true background image 174d (FIG. 1A), as it may be derived by subtracting the reflection background image 174b from the composite background image 174c.

Note that the description provided in FIGS. 1A and 1B can be generalized to handle rotation, and that derived models, such as image 178a can potentially be defined by either 3D images containing (3D) voxels, or 2D images containing pixels. Hence, while various embodiments and descriptions reference a 2D image space, it is expressly contemplated, and one of skill will recognize, that the principles therein can be extended to 3D imagery and/or with 3D models informed by multiple 2D images and/or image projections.

The images generated by the light segmentation module 173 are transmitted to the selective amplification and tone-mapping module 175. The amplification and tone-mapping module 175 selectively amplifies the subject image 174a. Further adjustment of the subject image is required because, when imaging an object with a bright reflection background component, the changes in image intensity associated with the subject are (typically) a very small fraction of the overall light/signal that reaches each image pixel. So even after the reflection background component 174b has been removed, both the magnitude and the range of the subject image's pixel values remain relatively small in comparison to the overall range of pixel intensities that were present in the original image 172. To make the subject visible, a combination of level shifting, amplification, and tone correction is desirable. Note that additional processing beyond image segmentation can often be desirable. Typically, the subject image signal resulting from image segmentation is extremely small, as it was just a small fraction of the reflection+subject signal that was captured by the camera/acquisition device 171. Even with the reflection component removed, the subject signal 174a is still very weak, and in many cases will contain negative intensity values. Tonal mapping, including scaling and level shifting, can be desired to make the subject more visible.

Referring again to FIG. 1A, the procedure 170, also performs amplification, level shifting, and/or tone adjustment on the subject image, and then using the resulting enhanced-subject image as input to an HDR compositing process. Illustratively, the image is amplified and then level-shifted upwardly, so that the image's minimum value is (e.g.) zero (0). In alternate embodiments, the subject layer intensities can be amplified by a factor of tens (e.g. 70×, 80×, etc.), with or without (free-of) color tone-correction and/or contrast-enhancement. Applying spatial operations and thresholding to the subject image can further refine which pixels should be amplified and which should be ignored. For example, since many subjects of interest (e.g. anatomical features) are typically more than one pixel in size, a spatial thresholding mask 174e can be used to remove isolated pixels. The procedure can also be informed by the true background 174d and reflection background 174b images as well. Specifically, thresholding the absolute value of the difference between the subject image 174a and the true background image can further refine the location of the subject by identifying locations where the subject occludes the true background. Applying selective amplification and tone correction to the subject image locations only, in regions where this difference from true background exceeds a threshold, is a desirable.

Results of the selective amplification and tone mapping module 175, which can include the processed subject image 176a, processed true background image 176b and subject location mask 176c (which can be similar or identical to the subject location mask 174e, described above), are transmitted to the optical property adjustment module 177 to correct color shifts, spatial distortions, and other effects of light/radiation passing through an occluding surface (for example, a semi-transparent surface such as a piece of tinted glass, or tissue/blood that obscures or occludes the subject feature(s). For example, in the specific use case of a window with a known tint gradient of a particular color, the color of the subject image can be shifted by the module 177 to compensate for the color-shift and intensity-shift introduced by the window at each location in the image. The magnitude of this color-shift and intensity shift can be measured directly, for example by placing a calibrated color card within the vehicle in front of the subject, and observing the difference in the subject image relative to the ideal known coloration of the card. Alternatively, the magnitude of this color-shift (in the above-described, visible-light example) and intensity shift may be known in advance, such as by knowing the inherent characteristics of the underlying subject being imaged (e.g. the make and model of a vehicle and the factory-provided window tint, or a particular piece of anatomy that has a known effect on intensity). Object identification techniques, or simple human-input (e.g. mouse-clicking on the corners of the windshield, or the perimeter of the anatomical feature, in a displayed image), can then be used to identify the boundaries of the window, from which the optical properties at each pixel can then be derived based on the known spatial gradient of the glass. If optical transmission properties are not available, the user can be presented with several possible correction settings, corresponding to common products likely to be encountered, and can choose among them based on preferred appearance of the enhanced subject image. Optical property adjustment can be advantageously applied to just the subject image, or to both the subject and the true background image and/or reflection background image, depending upon the needs of the application. For example, to image an outdoor scene using an indoor camera through a tinted window, it would be desirable to perform optical property adjustment on the true background as well as on the subject images, so that the scene background is corrected for the window tint color. However, in this example, it would (typically) not be desirable to perform optical property adjustment on the reflection background, since the reflection background would typically represent indoor light sources that it is not desirable to include in the resulting HDR image. It should be clear to those of skill that the parameters and adjustments of the optical property module could be modified to adapt to the specific characteristics of a medical image where optical properties are not generally considered, but ultrasound and/or electromagnetic characteristics reflections, distortions, etc. may be present.

For example, in the case of an x-ray image, in which a tumor is obscured by a rib, a model of the rib's radiographic properties can be created based on periods when motion causes the tumor to not be present in a region, or the model can be constructed based on pre-treatment imaging (e.g. accessed from storage 165). Analogous to the above-described optical property adjustment used to correct for tinting of glass, radiographic property adjustment (primary intensity amplification) can be applied to account for the reduced illumination of the tumor due to the overlying rib. In imaging arrangements, in which a rib underlying a tumor is to be visualized, radiographic intensity adjustment can be applied to account for intensity changes caused by the reduced effective illumination that results from the beam having to pass through the tumor, or potentially through both an overlying rib and the tumor to reach the underlying rib. Advantageously, this model of the rib radioopacity can be represented in 3D, and perspective-corrected, for the current position of the radiation source and/or detector.

The optical property adjustment module 177 generates enhanced images, including an enhanced subject image 178a, enhanced true background image 178b and the subject location mask 178c (which can be masks 174e and/or 176c, as described above). Images (178a, 178b) produced by the amplification and tone-mapping module, as well as images (174a, 174b, 174c, 174d) produced by the optical property correction module, can be passed back to the image acquisition module (via procedure branch 179) to provide feedback using an acquisition parameter optimizer 180, which informs adjustment of acquisition parameters. For example, adjusting focus of the camera so as to optimize the sharpness of the enhanced subject image. Similarly, adjustments to field strength, operating frequency, etc., can be applied in a medical imaging embodiment.

The enhanced image data 178a, 178b, and subject location mask 178c are transmitted to an HDR image composition module 182. This module 182 combines subject-enhanced images 178a with one or more traditionally-exposed images 183, so as to provide an overall view of a scene that places the subject in context. For example, a traditionally-exposed view of an automobile can be overlaid with a subject-enhanced view of a passenger in that automobile. This image fusion can be performed by the module 182 through a combination with the original, unprocessed image, or in an illustrative embodiment, through a combination of the enhanced subject image 178a with the true background image 178b and/or the reflection background image 174b. This module 182 also combines different enhanced subject images. These different enhanced subject images can be acquired/captured at the same moment in time, but have different degrees of level-shifting and amplification. For example, an image of a passenger in a vehicle can have very different lighting and require a different level of enhancement processing than an image of the driver of the vehicle. Treating these subject images separately for enhancement purposes, and then fusing the most visible aspects into a single composite HDR image, is a function of the HDR module 182. Advantageously, the subject images can be captured at different points in time—as is the case in many medical imaging application. In the exemplary use case of an automobile and its passengers, if two occupants of a vehicle sit still for an extended period of time, they will begin to be considered to be background light in acquired images. Then, if the driver of the vehicle starts moving, and turns his/her head towards the camera at time 3, he/she will create a subject image that is highly visible relative to the background. If the rear seat passenger then turns his/her head at time 10, he/she will create a similarly highly visible image. By merging together the images from time 3 and time 10, together with a traditionally exposed image of the vehicle, a composite view of the vehicle and its occupants can be produced. This is the output HDR image 184 of the procedure 170.

For example, in the case of radiographic imaging, with beam intensity optimized to see through a rib, detector saturation can occur in regions that the rib is not present, resulting in a black image with no features. As in the exemplary case of an automobile occupant that is only sometimes visible, an accurate picture of the saturated region can be acquired at a time when it is not saturated, i.e. when the rib is present. Compositing these views acquired at different moments in time can dramatically improve the ability to visualize hidden features such as tumors.

Many techniques for performing HDR image fusion and tonal mapping continuity are known to those of skill in the art of machine vision and associated literature. Many are based on optimizing the contrast of the image, combining images taken under different exposure conditions. Some recent work has focused upon HDR in video images, primarily from the perspective of ensuring continuity of the HDR merging process across multiple frames, so as to avoid blinking or flashes introduced by changes in HDR mapping decisions from frame to frame. However, the above procedure also advantageously teaches optimization of the HDR image tonal-mapping process based on motion of a subject relative to its background image.

More particularly, the illustrative embodiment can utilize the above techniques to separate out various different anatomical components that have been mixed together/confounded in the proton beam image sequences. In particular, it is thereby possible to disambiguate ribs from tumor from other objects such as blood vessels, based in part on the way that these features move around as the patient breathes, shifts position, pumps blood, etc., in image sequences acquired during proton beam (or similar radiation) therapy. Since the image portions due to the ribs change and/or move at a different rate from those due to the tumor, which in turn, change or move at a different rate from the image portions due to the blood vessels, it is possible to use the differential in motion rates and/or differential in the rate of change of each pixel's intensities, to estimate how much of the energy captured at each pixel is due to the tumor, how much is due to the ribs, and how much is due to blood vessels and/or other structures. The energy associated with an object of interest, such as a tumor, can then be selectively amplified or isolated. Other queues, such as brightness and texture, can be utilized as well to further disambiguate the various anatomical structures that contribute to the confounded image. Finally, techniques such as amplification and contrast stretching, are employed prior to motion analysis, to make the motion more visible, and also following motion analysis to selectively enhance the portion of the energy that is associated with the object of interest.

Once the object/subject of interest within the image is isolated, characteristic information about its position and spatial extent can be extracted using standard computer vision techniques/tools, such as blob detection and feature extraction. The resulting characteristic measurements can then be utilized to enhance the measurement of radiation dosage (dosimetry) delivered during proton beam or other types of targeted therapy, or more basically, the total exposure time of each piece of tissue (distinct from dosimetry in that radiation dose is both depth-dependent and tissue-type-dependent), and can also be used to optimize the delivery of the radiation dosage itself in real-time through the techniques described below. In addition to proton beam therapy, this approach can be utilized in other forms of targeted therapy that require accurate tracking of anatomical objects and targeted energy delivery to a localized area, such as ultrasonic ablation of tissue.

As also described below, the extracted measurements can be used to fit a multi-dimensional model of the patient's anatomy to the imaging data. This model can be augmented by data collected via conventional three-dimensional (3D) or four-dimensional (4D—three dimensions plus time) MM or CT imaging performed prior to radiation treatment. In particular, synthetic projections derived from the multi-dimensional MRI or CT imaging can be compared with the actual data obtained during treatment to infer information about the position of anatomic features relative to the treatment beam.

III. Operational Embodiment

In an illustrative embodiment, and as shown in FIG. 1, a lung cancer tumor is detected during proton beam therapy by real-time imaging acquired using the radiation produced by the proton (more generally termed “radiation” or “treatment”) beam 114, itself. The FPI (also termed a “detector”) 140 mounted on the opposite side of the patient 130 from the source of the radiation captures an image that provides a projection view of the patient's anatomy to the processor 160, as defined by the path of the radiation beam. This image is then analyzed by the modules of the processor 160 to determine characteristics of interest such as tumor location. Notably, the timing of the firing/activation of the radiation beam 112 is limited by module 166 to occur primarily when the computer vision algorithms (module 164) determine that the tumor is within the beam's path. However, the beam may also be fired when the tumor is outside the desired location to discern its current, relative position. The beam's energy is cut off/deactivated when patient motion (for instance due to respiration) moves healthy tissue (rather than tumor) into the path of the beam. Note, while a proton beam is shown by way of example, the above-referenced terms “treatment beam”, “radiation beam”, or similar, can be substituted, as the principles herein are applicable to a wide range of radiation-based treatment regimes, in which imagery can be acquired during the process so as to assist in diagnosing the condition being treated and/or guiding application of the treatment (e.g.) in real-time. Other forms of treatment radiation can include, but are not limited to gamma ray treatment (via a gamma source, etc.), various x-ray treatments, stereotactic body radiation therapy (SBRT), three-dimensional conformal radiation therapy (3D-CRT), intensity-modulated radiation therapy (IMRT), radiosurgery, and other forms of treatment, that should be clear to those of skill.

In an alternate embodiment, the beam is itself is steered (by the shaping/positioning assembly 116) in real-time using feedback from the module 166 to keep the tumor centered or to keep the tumor's overall boundary within the beam's target radiation pattern. While steering can be accomplished electronically using electromagnets, in a further alternate embodiment, it is contemplated that the shaping/positioning assembly 116 can include an electromechanically actuated, spatially deformable shutter (typically an array of tungsten or brass flaps) that is adjusted in real-time to adjust the size and shape of the irradiated area to match the spatial extent of the tumor. In essence adjusting the ‘spot size’ of the beam. This spot size adjustment can also be used independently of, or in combination with, the beam timing and/or steering approaches.

In another alternate embodiment, the support table (163 in FIG. 1.) on which the patient is positioned is actuated (for example through a pneumatic or hydraulic cushion), or another appropriate actuation system 161 that is in operable communication with the processor module 166, so as to move the patient along one or more axes 169 (degrees of freedom) in a manner that keeps the tumor located within the target area of the radiation beam. For example, as the patient breathes in, an inflatable pad on the table deflates slightly, to compensate and keep the tumor's position stationary relative to the treatment beam.

In an alternate embodiment, the treatment (e.g. proton) beam emitter and flat-panel detector can be mounted on a gantry and rotated around the patient so as to provide irradiation and imaging from a plurality of angles relative to the patient. The data gathered from these multiple perspectives are combined to form a multi-dimensional model to estimate the location of anatomic features.

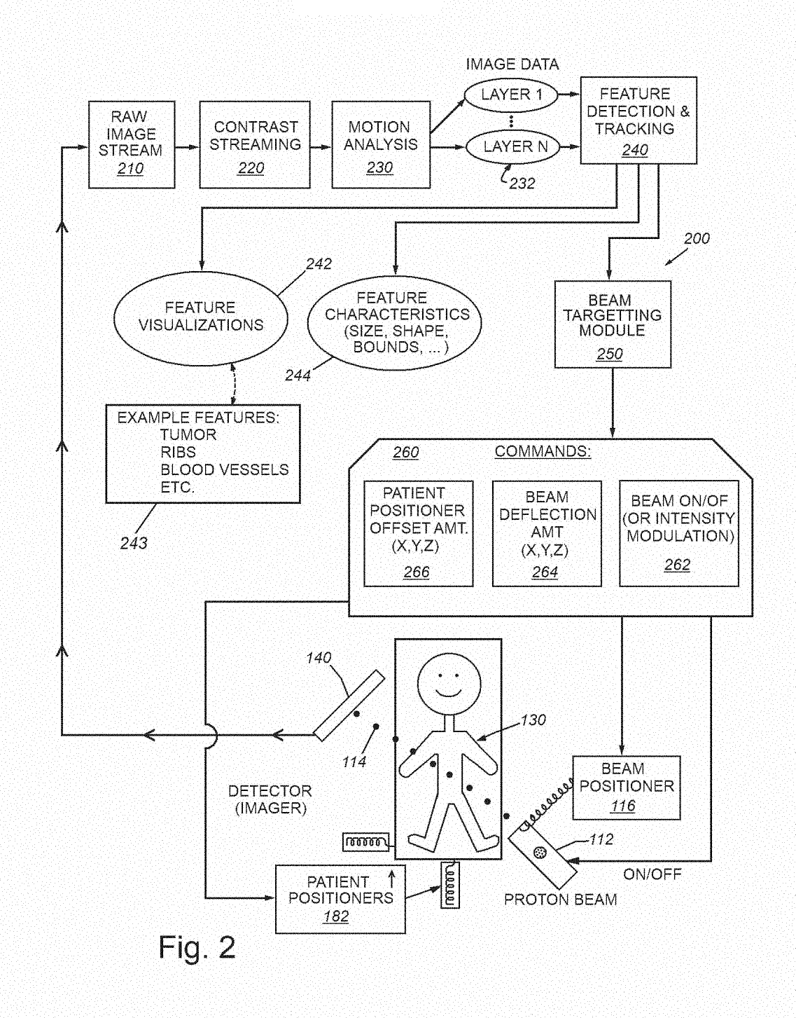

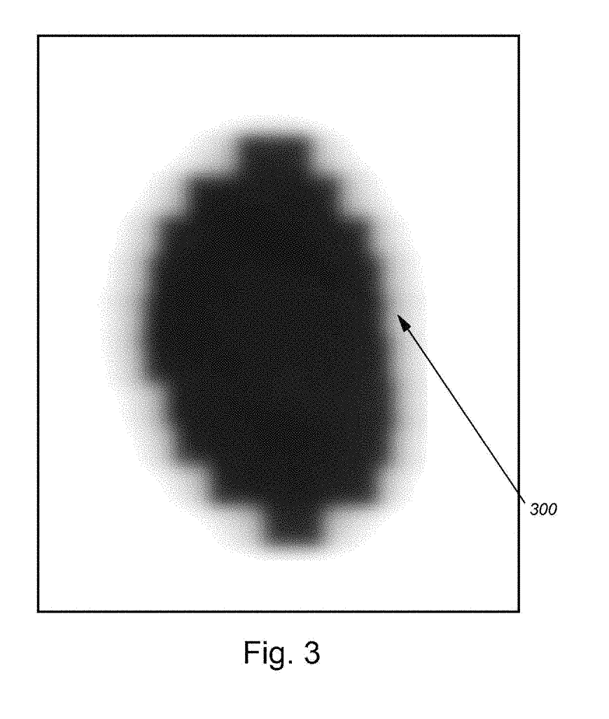

With further reference to the block diagram of FIG. 2, the overall procedure 200 is shown and described in further detail. As described above, the treatment beam device 112 irradiates the patient 130 with the beam 114, which is received by the FPI (detector) 140. The beam 114 is directed by the positioner 116 so as to vary its path with respect to the region of interest within the patient. Additionally, or alternatively, the patient can be moved variably via the positioner actuators 182 to achieve desired alignment of the beam 114 with the region of interest. Data from the FPI 140 is transmitted as a raw image stream 210. An example of a raw image 300 is shown in FIG. 3. The features in this image are largely undifferentiated, making it difficult, or impossible, for a user or automated vision process to discern bone, from blood vessels and/or tumorous tissue.

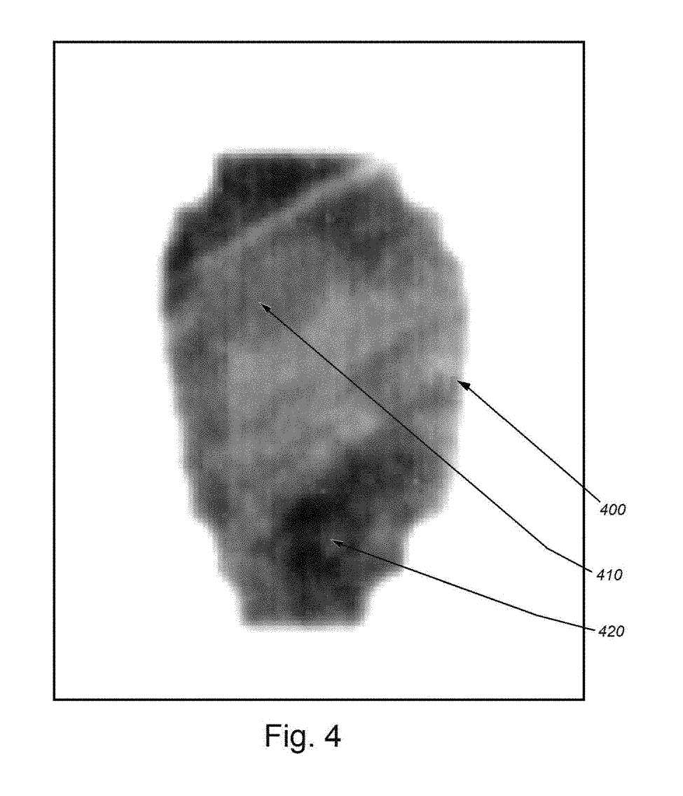

The raw image stream 210 is then subjected to a contrast stretching (mapping) process 220 to derive features that are more discernable. The results are depicted in the image 400 of FIG. 4, with certain lighter areas 410 representing (e.g.) tumorous regions. Very light areas 420 can represent bone and other types of tissue. In alternate examples, the light and dark contrasting regions can represent different types of tissue.

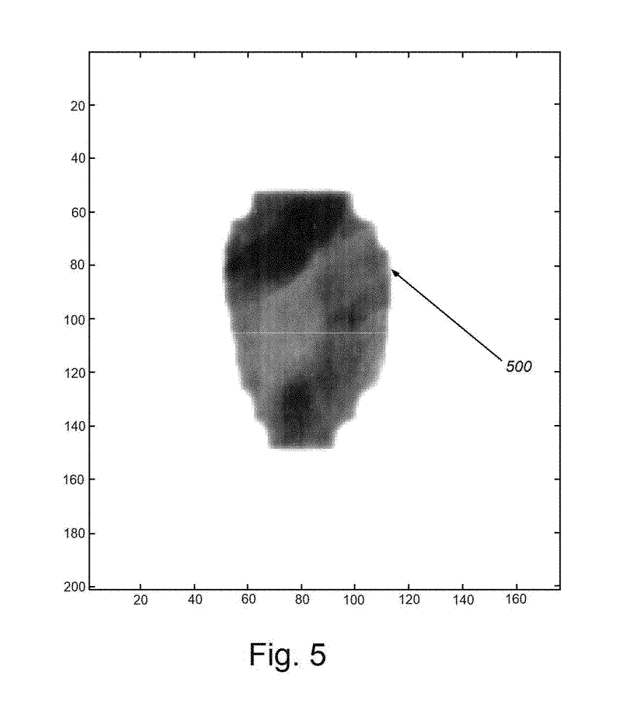

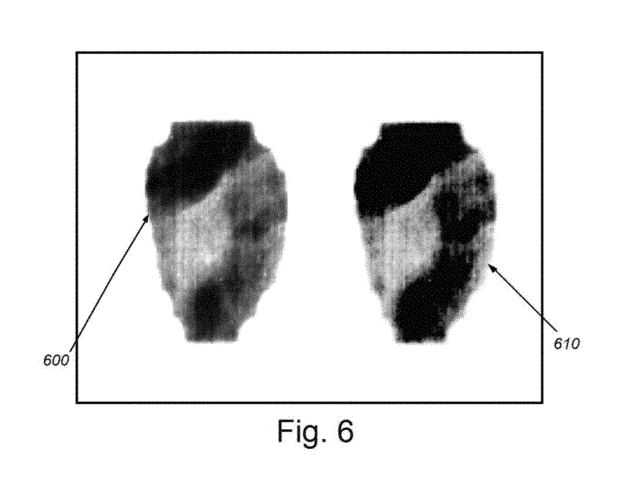

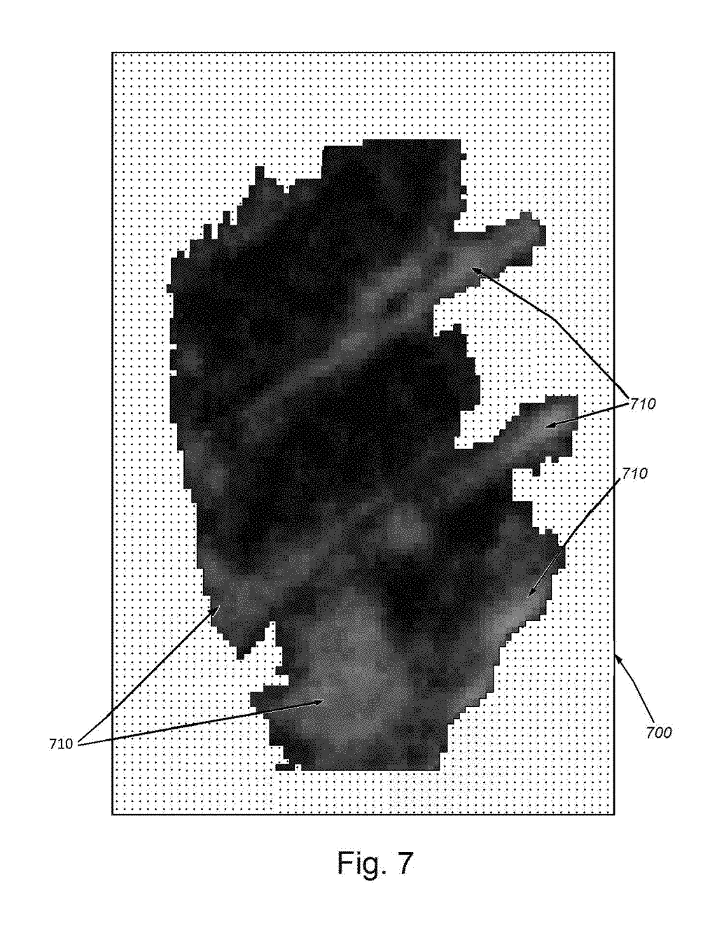

Following contrast stretching, which allows features to be resolved within limits, the image is then subjected to motion analysis (process block 230) to derive layers (1-N) 232 based upon perceived motion. This motion is represented by change in intensity over time, where a plurality of image frames are each compared to a reference frame. FIG. 5 illustrates the fraction of the treatment beam image 500 whose intensity does not vary (is free of variation), i.e. the minimum intensity present in each element of the treatment beam image stream. With further reference to FIG. 6, the image 500, representing, the fraction of the treatment beam image whose intensity does not vary is shown as an enhanced image 600 on the left after undergoing local contrast enhancement. On the right, the same image 500 (i.e. the fraction of the treatment beam image whose intensity does not vary) is shown as an enhanced image 610 after undergoing constraint-based subtraction to adjust contrast, in which a constraint is manually applied indicating that the darker material that has a different texture is to be ignored. In operation, this constraint can be derived automatically based on detection of the differing motion of the tissue texture between the dark areas and the light areas—e.g. by detecting differences between image frames over time. This property is illustrated in FIG. 7, showing a typical image frame 700 of the portion of the treatment beam image whose intensity does vary significantly, i.e. the temporally dynamic portion of the image. By way of example, blood vessels and other dynamic features 710 become apparent and can be used for automatic derivation of the above-described constraint.

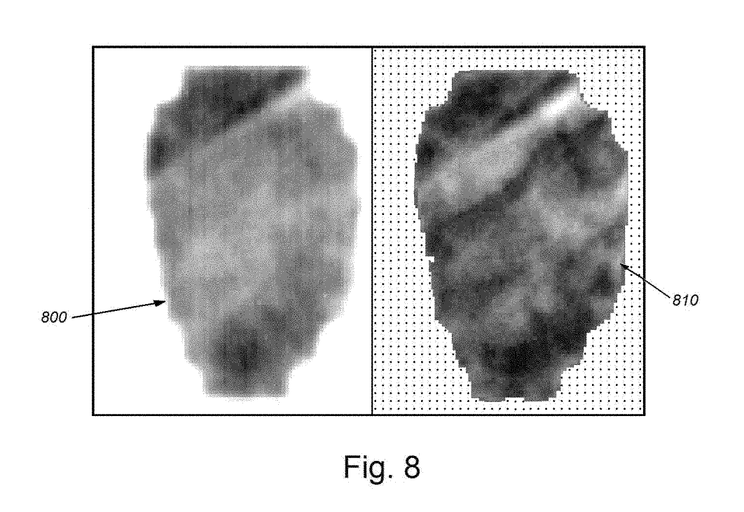

Referring to FIG. 8, on the left the full image frame 800 that served as the starting point for the motion-based processing (process block 230), having undergone only basic contrast stretching (process block 220), is shown for reference. On the right, an image frame 810 is shown from a blended video that amplifies the dynamic portion (derived e.g. from image frame 700) but also includes 30% of the static portion, for reference. This blended view is significant because human observers are not used to viewing dynamics completely independent of the static background. However, the depicted frame 810 makes the dynamic features more visible while also showing the static background. In essence, the use of the dynamics in the process lets one see through the cloudiness. Notably, when compared to the nearly featureless raw image 300 of FIG. 3, the enhanced and feature-rich image 810 is substantially more meaningful to a user or automated process.

The features of the image 810 can be subjected to vision-system based feature detection and tracking in process block 240. A variety of conventional tools, such as edge/contrast detectors, blob analyzers, and others clear to those of skill can be operated. Such tools are commercially available from a variety of vendors. The results of feature detection can include visualizations 242 of the features in the form of (e.g.) tumorous tissue, ribs/bony structures, blood vessels, other soft tissue, nerves, etc. 243. The detection and analysis of features in the image can also provide the discrete feature characteristics 244, such as size, shape, boundaries, etc. Various shape-fitting tools, pattern matching tools, caliper tools, etc. can be employed to obtain these results, in a manner clear to those of skill.

The feature detecting and tracking module/process 240 also transmits feature information to a beam targeting/position module/process 250. The feature information can include identification of fiducials within the image that can change position between frames. The targeting module establishes a coordinate system (for example, Cartesian coordinates (orthogonal x, y and z axes), or polar coordinates) with respect to the image, and the fiducials are located in the coordinate system. Movement between image frames is tracked and translated into motion vectors. These vectors can be used to generate various commands 260 for controlling the beam via the beam positioner 116. For example, the tracked motion can result in an on/off command, or modulation of the beam intensity 262 to ensure it does not strike unwanted regions of the tissue. The motion can also be translated into an appropriate degree of beam repositioning 264 using the positioner 116 (and employing magnetic and/or electromechanical beam deflection). Alternatively, or additionally, the motion can be translated into commands 266 for the patient positioning actuator(s) along one or more axes (and/or rotations, where appropriate).

Note that it is also desirable to track non-tumor objects such as ribs. In this manner, radiation dosage can be limited to times when the ribs are present with respect to the beam path, so that in situations in which the radiation beam energy was selected based on its need to penetrate the rib, the radiation exposure is not provided (i.e. being free of exposure) when the rib is not present in the treatment field. This prevents over-exposure of the patient to high-energy radiation. Similarly, in cases where the treatment energy level is designed to be lower energy based on the expectation that the rib would not obscure the tumor from exposure to the radiation, the radiation can be withheld when the rib is obscuring the tumor, thereby reducing exposure of the patient to radiation at a time that the radiation would be ineffective due to the rib occlusion.

IV. Image Processing and Enhancement Steps

The above-incorporated application and subject matter operates to separate the acquired image into layers and enhance that image so that features are more visible and defined using the steps generally described in accordance with the above-incorporated Application and subject matter of the above-referenced procedures and related matter described in FIGS. 1A and 1B, including published U.S. Application No. 2017/0352131 A1. Briefly, the processor employs an adjustable image acquisition parameter. This parameter is adjusted based on an image that has had its subject selectively enhanced via comparison with at least one background reference image. More particularly, the process identifies background portions of the original image and obscured (for example, by reflections as described in the description of the above-referenced procedures and related matter of FIGS. 1A and 1B, and published U.S. Application No. 2017/0352131 A1) portions of the original image. The process also identifies second, non-stationary portions of the original image. The process then identifies a subject/feature(s) of interest portion of the original image. In this case, at least some of the obscured/reflective portions or the non-stationary portions overlie the subject/feature(s) of interest. Selective amplification is applied to the background portions, obscured/reflective and non-stationary portions, and the subject/feature(s) of interest portions of the image. Additionally, level shifting is applied to the subject/feature(s) of interest portions. Then, tone correction can be applied to the subject/feature(s) of interest to provide an enhanced image of the subject/feature(s) of interest—in this case delineating blood vessels and associated tumorous tissue from surrounding tissue and bone. The application of selective amplification can be achieved by a user operating a user interface (UI) or via automated processes within the processor 160. Likewise, level shifting and tone correction can be achieved by automated mechanisms—using (e.g.) default or custom parameters to apply the appropriate level of correction.

According to another aspect of the procedure, the image of the subject of an original image is enhanced using a background module that identifies background portions of the original image, which include stationary portions of the original image. A first reflection module identifies first reflective portions of the original image that move slower than a first rate, r1, over a first period of time. A second reflection module also identifies second reflective portions of the original image that change faster than a second rate, r2. The rate r2 is faster than the rate rl over a second period of time. A subject/feature(s) of interest module is provided for identifying a subject/feature(s) of interest portion of the original image. In this case, at least some of the first reflective portions or the second reflective portions overlay the subject/feature(s) of interest portion. The processor is arranged to selectively adjust the amplification and tone adjustment of the original image to provide an enhanced image of the subject/feature(s) of interest.

According to another aspect of the procedure, the system maintains a model of the portions of the image that can be momentarily occluded by features, such as ribs entering the field of view, or by over-exposure caused by ribs exiting the field of view causing the detector to saturate. These modeled portions may then be displayed as a reference to help physicians or automated software locate features of interest, such as a tumor, even when they are momentarily not visible in the current frame of acquired image data. This is akin to the mosaicing concept described for use with visualization of vehicle occupants in the above-referenced procedures and related matter of FIGS. 1A and 1B, and published U.S. Application No. 2017/0352131 A1.

According to yet another aspect of the procedure, computation of change rates and other intensity value statistics (such as minimum and weighted average) are employed to differentiate the various layers can be restricted to intervals for which the detector is not saturated, and the element of interest is not occluded. In an exemplary embodiment, the MINIMUM INTENSITY PROJECTION can be utilized to estimate the portion of the composite background that is due to the reflection background, since the reflection background is present even as the subject moves around the image field. To prevent saturation (which results in a solid black image) from forcing the MINIMUM INTENSITY PROJECTION's value to 0, the projection is computed only for those pixels of each frame for which detector saturation is not present. Saturation can be detected as an image intensity that lies below a cutoff threshold near 0 (black). Advantageously, the values of saturated may be estimated using values of neighboring non-saturated pixels, using a model of tissue motion based on previous observations, or via interpolation between non-saturated signal measurements from nearby time points.

V. Additional Arrangements and Functions

A. Wearable Device

In an alternate embodiment, a wearable deformation device, such as a pneumatically-actuated girdle, is utilized in conjunction with tracking information to apply deformation forces to the patient that act to keep the tumor position centered within the target area of the treatment (e.g. proton) beam. For example, if the tumor is located on the right side of the body, as the patient inhales, pressure can be applied to the right side of the chest to modify or restrict inflation and/or motion of the ribs on the right side of the body, in a manner that minimizes tumor motion while still permitting the patient some degree of ability to breathe.

B. Fusion of Pre-Treatment Scanned Imagery

In another alternate embodiment, it is contemplated that the (2D) images that the above-described approach is exposing in the treatment beam imagery can be correlated against the pre-treatment imaging (e.g. 3D or 4D CT, MRI or PET) scans of the same patient (e.g. from source 167), to identify the features that the analysis of the 2D image sequences provided by the treatment beam process is making more visible. More particularly, the 2D image features can be compared for the accuracy of their correlation with known anatomical features in the 3D CT scans to determine if certain types of features are more accurately imaged than others using the illustrative approach. In other words, to ascertain the extent to which the features that are viewed as a tumor in the 2D scans are, in fact, the full tumor as visible in the 3D scans. From this determination, safety margins can be derived so that the treatment beam is not targeted too narrowly on the visible portions of the tumor. More generally, these medical imagery-based, pre-treatment images are provided to an analysis module that generates fused images. The fused images are a combination of pre-treatment (pre-operative) images and enhanced image frames from the treatment beam imagery. The fused images thereby provide the user with depth and/or information relative to the features. This can assist in identifying the features, and their location (depth) when the displayed images from the treatment beam are unclear or ambiguous as to such information.

C. Arm-Mounted/Rotating Treatment Beam

It is contemplated that the treatment beam (proton, etc.) can be mounted on a continuously rotating treatment arm or similar structure that includes a continuously moving aperture whereby the patient is encircled by the treatment arrangement with the beam remaining focused on an internal region of the body and passing around the exterior thereof. The detector can be arranged as a ring, and/or as a discrete member that rotates similarly, and on an opposite side of the patient from the emitted beam. This optional rotation is represented in FIG. 1 by the arcuate dashed-line arrows 159. As such, the beam emitter effectively encircles the patient to emit the beam around a 360-degree perimeter. This allows for a more complete 3D view of the region of interest and also avoids passage of the beam through one collimated portion of the body for too long an interval—hence reducing that particular region's exposure to potentially harmful treatment radiation. While not shown, the arm or other device that provides rotation to the beam can transmit position information—e.g. based on a stepper motor pulse stream, encoder, etc.—to the processor 160 (in a manner clear to those of skill), so that its location and rate of rotation are tracked. This can assist in determining where a particular image frame is acquired with respect to the overall geometry of the patient and treatment region. This position information can be used to inform the image fusion process as to the perspective from which the 2D image was acquired, permitting higher quality fusion with pre-treatment imagery, and appropriate perspective correction of motion/change information considered by the layer separation processor 162.

D. Display of Layer-Separated Images

While the above-described embodiment enables automated control of the treatment beam (e.g. steering, intensity, etc.) using the beam control processor 166 and its interactions with the layer separation processor 162, vision tools 164, and other operational components, to operate the beam controller and steering 118, it is expressly contemplated that the system can deliver layered images (via the display processor 168) to the computing device 150. These images can highlight features in the region being treated and allow the practitioner to manually control the beam in real-time. The practitioner/user steers the beam via an interface—for example provided on the computing device 150, or a separate control console (not shown).

In addition to the use of the system to target therapy by automatically guiding the beam, or allowing the practitioner to manually guide the beam (control being represented by data block 157 in FIG. 1), as described above, the multi-layer separation techniques described herein that are applied to monitoring of the radiation beam can also be used to provide feedback to the practitioner about which tissue has been exposed to the treatment beam. This feedback can take the form of an image or video that shows the practitioner an enhanced photographic representation or delineation of an object of interest, such as a tumor, without undue interference from surrounding tissues. This can be achieved by preferentially amplifying the object of interest relative to the intensities of other signals present in the acquired image. Alternatively, this enhancement may be achieved by introducing artificial colorization, such that the object of interest is more readily distinguished from overlying or underlying tissues. This feedback can also (or alternatively) take the form of an indication of the total duration of exposure to the beam that each layer has undergone. For example, in a situation where each piece of tissue is irradiated multiple times, or from multiple angles, due to breathing, different pieces of the tissue of interest will be present in the beam's path at different times. Tracking the time-in-path of the beam of each piece of tissue can inform the practitioner as to the effectiveness of the radiation delivery, and whether additional passes or changes to the aperture are desirable. Tracking the depth (distance from radiation source) at which that exposure occurs can also be very useful in estimating radiation delivery to specific pieces of tissue. The indication of total exposure, optionally further categorized by depth information, can be characterized by (a) color-coding of an image, (b) displaying of a graph of exposure history in response to selection of a location of interest via a mouse pointer or cursor, and/or (c) other graphical indicators for representing exposure. This is represented generally in FIG. 1 as a series of display outputs 193, including, but not limited to, layered images 194, colorized and/or colorized imaged (or regions thereof) 195, histograms and/or other data plots 196 and other forms of graphical output that can include image features combined with other synthetic or actual image data 197. Tracking can be performed using conventional feature tracking algorithms/processes, applied to the layer-separated images 174 or 178.

Since depth, as well as total exposure time, is desirable in determining radiation dose applied to the treatment region, use of pre-operative imaging (such as CT, ultrasound, or MRI scans 167, as shown in FIG. 1) can be employed to track the depth at which exposure has occurred. For example, in the case of a beam that rotates around a patient (arrows 159 in FIG. 1), the tumor will be located at different depths depending on the current rotational position of the beam emitter 112. By tracking total exposure time, as well as depth information, a histogram (or other graphical representation—e.g. a plot) of exposure time vs. depth can be generated (e.g. by the display processor 168) for each location in the layer of interest.

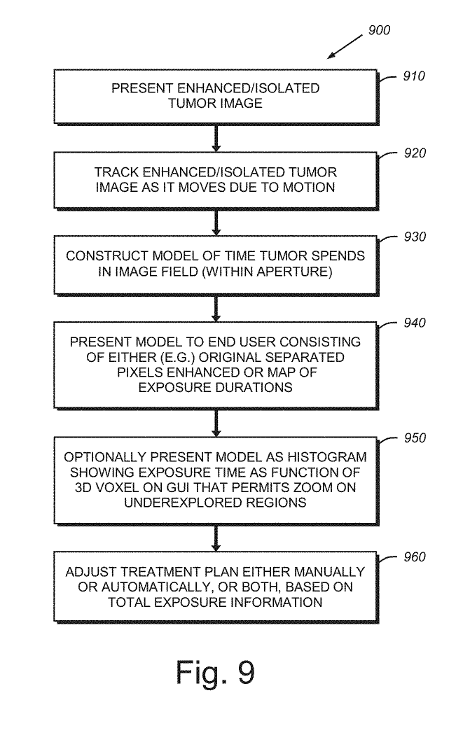

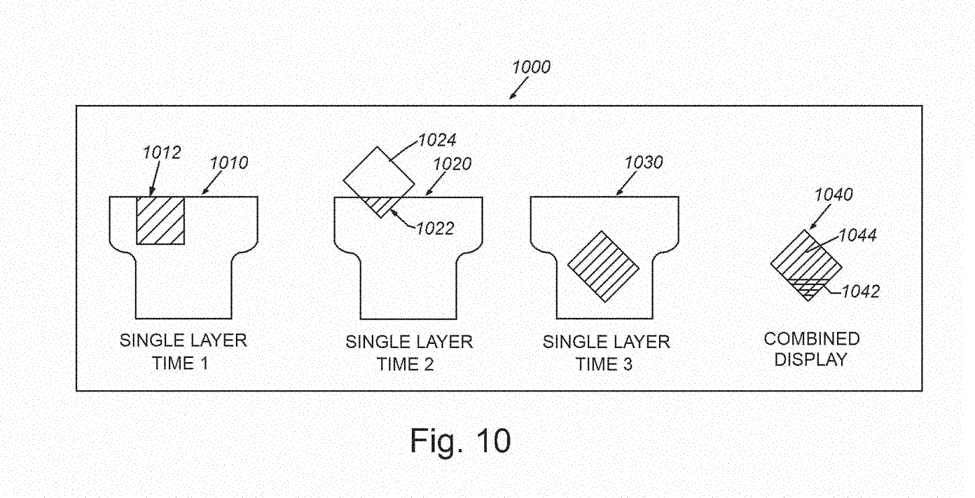

Reference is now made to FIG. 9, which shows a block diagram 900 relative to modeling imaged tissue, and FIG. 10, which shows a diagram 1000 of exemplary image frames, acquired during a period of patient motion. It is contemplated that presenting a model of tissue of interest to the user, even without modifying the radiation treatment plan in real-time, can be valuable in planning the overall diagnostic and treatment regime. For example, the target exposure levels of each piece of tissue in a later imaging session can be informed by the model of tissue exposure acquired during previous imaging sessions. In that manner, over multiple exposure sessions, the practitioner can ensure that the entire tumor region is adequately irradiated. Similarly, compiling an estimate exposure of surrounding tissue, such as portions of ribs can help inform future session treatment plans, in which exposure can be initiated at a moment in the system's rotation or the patient's position, which thereby avoids a piece of non-tumor tissue that has undergone significant radiation exposure in previous treatment sessions.

In the block diagram 900, an enhanced/isolated tumor image is presented by the system. This image is separated from ribs and other obstructions (block 910). The system tracks the enhanced/isolated tumor image as it moves in and out of the treatment field due to breathing and/or other patient motions (block 920). The system constructs a model of the time the tumor spends in the radiation field, within the aperture (block 930). Optionally, this modelling can be augmented with the depth of the tumor relative to the current position of the beam emitter (which can be useful in estimating radiation dose in addition to total exposure time). A model is presented to the end user (practitioner or treatment specialist), which consists either of the original separated pixels that are enhanced, for example via color coding, to represent total exposure time in a basic embodiment, or in another embodiment, estimated radiation dose (block 940). Alternatively, rather than displaying the original separated pixels, a map of exposure durations can be presented. This arrangement is shown further by way of example in the diagram 1000 in FIG. 10 (described below). Alternatively (optionally), the model can be, for example, presented as a histogram showing exposure time as a function of 3D voxels, together with a graphical user interface (for example, GUI 156 in FIG. 1), which permits the user to zoom in on underexposed regions (block 950). Notably, the system provided in the diagram 900 allows for adjustment of the treatment plan (exposures to perform by the treatment beam in a stationary or moving/rotating arrangement), either manually or automatically, based on this total exposure information (block 960). Treatment adjustment can be performed in real-time, or can be performed during a later part of the same treatment/exposure session (e.g. a few minutes later). Alternatively, it is contemplated that such adjustment can be reflected in future treatment sessions (another day/ beam exposure cycle). This adjustment can compensate for temporal and/or physical variations in exposure to ensure that, over time, the entire tumor receives an adequate amount of exposure to the treatment beam.

As described above, with particular reference to the image frame display 1000 of FIG. 10, which shows a series of image frames 1010, 1020 and 1030 acquired from a patient body in a treatment region at discrete times. Each represents a single layer of a (e.g.) 3D image generated by treatment beam exposure. In the first time (Time 1) frame 1010, the target/treatment region 1012 is shown fully imaged and exposed to treatment radiation. This is indicated by the fully shaded target. Then, in Time 2, the target 1012 has moved partially out of the frame 1020 and only a portion 1022 (shaded) remains exposed to treatment radiation. This can be due to patient motion as a result of (for example) patient breathing, voluntary muscle movements, involuntary muscle movements, heartbeat and blood flow, or a combination of such factors. Thus, the unshaded portion 1024 of the target 1012 receives less radiation. Then, in the frame 1030 of Time 3, the target 1012 moves fully back into the field (again due to patient body motion). Thus, the entire target is shaded as it is fully exposed to the treatment radiation. The display process(or) 168, combined with other modules of the overall process(or) 160 generates the resulting combined display 1040, which essentially characterizes a 2D display of a 3D tissue model, of the target 1012. This display 1040 is color-coded or shaded so that the area 1042 is highlighted (cross hatched shading that can conform to a color or other indicia), as this area 1042 experiencing a longer exposure time that the remaining area 1044. More particularly, in this example, the highlighted area 1042 corresponds to the portion 1022 of the target 1012 from image frame 1020 (Time 2) that remained in the exposure field, while the remaining portion 1024 of the target 1012 left the field. This display can aid a user/practitioner in determining which areas of the treatment region may require additional exposure or to adjust the placement of the beam to ensure these underexposed areas are fully covered, and similarly, that an over exposed area subsequently receive less treatment exposure.

VI. Conclusion It should be clear that the above-described embodiments provide an effective and accurate way to apply radiation therapy and image the results thereof in a manner that reduces potential damage to healthy tissue surrounding a tumor or other target. The approach provided also generates highly useful and detailed images of the treatment field that can be viewed by a user in (essentially) real-time, and can be used by a variety of automated vision system processes to diagnose and control treatment. Likewise, the system and method provides an effective tool for monitoring, controlling and adjusting treatment (in an automated and/or manual manner) through separated image layers that can be displayed with various graphical data, such as color-coding, plots and other meaningful data. Additionally, the illustrative system and method differs from conventional 3D voxel visualization in that it presents results relative to tissue, rather than relative to a global 3D coordinate space. Thus, as motion occurs in the tissue, the system and method, adjusts the images to compensate for it, and the accumulated statistics associated with each location account for the tissue motion. Effectively, the system and method allows visualization of what has happened to actual pieces of tissue over time, even as they change locations, rather than simply visualizing acquired images. Moreover, in addition to the novel techniques of separation for feature tracking described herein, the system and method provides novel techniques for handling occlusion or saturation, including, but not limited to compositing of an image to fill in or replace features that may be missing (or inaccessible/unable to be acquired) due to saturation.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, also as used herein, various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 1-2%) of the system. Note also, as used herein the terms “process” and/or “processor” should be taken broadly to include a variety of electronic hardware and/or software based functions and components. Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor here herein can be implemented using electronic hardware, software consisting of a non-transitory computer-readable medium of program instructions, or a combination of hardware and software. Accordingly, this description is meant to be taken only by way of example, and not to otherwise limit the scope of this invention.

What is claimed is:

1. A system for applying a radiation beam for therapy of an internal region of a body of the patient that passes through the region and is received by a detector to generate images thereof, comprising:
an image processor that receives the image data from the detector as a plurality of image frames, and that performs layer separation within the plurality of image frames; and
a motion analysis module that compares static and dynamic features in the image frames to derive features in the separated layers, the image frames based on the layers of features being provided to an output as enhanced image frames.

2. The system as set forth in claim 1, further comprising a feature detection module that applies vision system tools to the features in the enhanced image frames to identify information contained therein.

3. The system as set forth in claim 2, wherein the information is used to track motion of the features versus time.

4. The system as set forth in claim 3, wherein the tracked motion information is provided to a beam positioner that changes a position or orientation of the radiation beam based on a degree and direction of tracked motion.

5. The system as set forth in claim 3, wherein the tracked motion information is provided to an actuation system that moves or restrains the patient to maintain the radiation beam at a desired position in the region.

6. The system as set forth in claim 1, wherein the radiation beam is at least one of x-rays, gamma rays, a proton beam, a stereotactic body radiation therapy (SBRT) source, a three-dimensional conformal radiation therapy (3D-CRT) source, an intensity-modulated radiation therapy (IMRT) source, and a radiosurgery source.

7. The system as set forth in claim 1, further comprising an analysis module that compares anatomical features from scans obtained at a time remote from the use of the radiation beam to features output in the enhanced image frames.

8. The system as set forth in claim 7, wherein the scans provide CT-based, MRI-based, PET-based, or other medical imagery-based, pre-treatment images and the analysis module is arranged to generate fused images comprising the pre-treatment images and the enhanced image frames.

9. The system as set forth in claim 8, wherein the fused images include at least one of information and depth relative to the features.

10. The system as set forth in claim 1, wherein the radiation beam is arranged on a continuously rotating structure that encircles the patient to emit the beam around a 360-degree perimeter thereof

11. The system as set forth in claim 1, further comprising a display processor arranged to display to a user a display model that is derived from the enhanced images, including information useful in diagnosing the imaged region or administering treatment to the imaged region.

12. The system as set forth in claim 11, wherein the information defines at least one of (a) shading and (b) color-coding of areas of the display model to characterize a degree of exposure to the radiation beam over time.

13. The system as set forth in claim 12, wherein the information is defined as at least one of (a) a graph, (b) a histogram, and (c) a plot that characterized exposure to the radiation beam versus time across the region.

14. The system as set forth in claim 13, wherein the display processor is arranged to perform contrast stretching on the plurality of image frames to assist in visually resolving image features therein.

15. The system as set forth in claim 1, wherein the radiation beam is arranged to rotate about the patient, and further comprising a tracking process that accounts for motion of the beam with respect to the region in generating the enhanced image frames.

16. The system as set forth in claim 1, further comprising a fusion module that integrates pre-treatment information with the enhanced images to assist in defining a subject of interest in the region relative to other information therein.

17. The system as set forth in claim 16, wherein the subject of interest is a tumor and the pre-treatment information identifies a layer of the enhanced images containing the tumor.

18. The system as set forth in claim 17, further comprising a pattern-matching process that operates on the pre-treatment information, in the form of pre-treatment images, and the enhanced images based upon matching, for at least one of the pre-treatment images and the enhanced images, at least one of (a) estimated volume, (b) shape, (c) texture, (d) intensity histogram, (e) edges, (f) velocity, and (g) projected area.

19. The system as set forth in claim 1, wherein the motion analysis module is arranged to identify and manipulate instances of occlusion or saturation in the plurality of image frames.

20. The system as set forth in claim 19, further comprising an image compositing process that is arranged to fill in items of the enhanced images that are missing based upon the occlusion or saturation.

21. The system as set forth in claim 1, further comprising an image processor that selectively applies at least one of amplification and tone-correction to the enhanced images.

22. The system as set forth in claim 1, further comprising an intensity adjustment process that compensates for intensity reduction in a subject image frame of the plurality of image frames based upon loss of signal energy due to items present in image frames overlying or underlying the subject image frame.

23. A method for treating and imaging a tissue region in a patient, using a radiation treatment beam that passes through the region and is received by a detector, comprising the steps of:
receiving the image data from the detector as a plurality of image frames;
analyzing static and dynamic features in the image frames to derive layer-separated images of the features; and
outputting the image frames based on the layers of the features as enhanced image frames.

24. The method as set forth in claim 23, comprising the step of performing contrast stretching on at least one of the layer-separated images to resolve features therein.
